(19) United States
(12) Patent Application Publication
(10) Pub. No.: US 2024/0415937 A1
(43) Pub. Date: Dec. 19, 2024

(54) THEOPHYLLINE-LOADED COLLAGEN NANOPARTICLES

(71) Applicant: KING FAISAL UNIVERSITY, AL-AHSA (SA)

(72) Inventors: MAI MUSTAFA KHALAF ALI, AL-AHSA (SA); HANY MOHAMED ABD EL-LATEEF AHMED, AL-AHSA (SA); AHMED MOHAMMED ABU-DIEF MOHAMMED, AL-AHSA (SA); AYA A. AWAD, AL-AHSA (SA); GABER ELSABER ABD EL-WANIS BATIHA, AL-AHSA (SA); MANAL ALY SHALABY, AL-AHSA (SA); HAZEM MOHAMED SHAHEEN, AL-AHSA (SA)

(21) Appl. No.: 18/210,873

(22) Filed: Jun. 16, 2023

(51) Int. Cl. A61K 38/39

(52) U.S. Cl. CPC A61K 38/39; A61K 31/522; A61K 47/50; A61K 47/6929; C07D 473/08; C07K 14/461; B82Y 40/00

(57) ABSTRACT

A nanocomposite is provided, the nanocomposite comprising collagen nanoparticles loaded with theophylline. The nanocomposite is generally spherical and can have an average particle diameter ranging from about 30 nm to about 40 nm. The collagen nanoparticles can be derived from a marine source, such as mullet fish. The nanocomposite can be useful for treating bronchial asthma.

### BACKGROUND

### Field

The disclosure of the present patent application relates to collagen nanoparticles, and particularly to theophylline-loaded collagen nanoparticles.

### Description of the Prior Art

Collagen is a structural protein which forms the soft tissues such as dermis, ligament, tendons, vessels and hard tissues such as bones. Collagen constitutes 6% of body weight of the mammals and 25-30% of all body proteins. Collagen is the protein which is most commonly found in the soft and solid tissues of the body such as tendon, ligament, skin, cornea, cartilage, bone, blood vessels, gout, namely gastrointestinal system, teeth, gums, vertebrae, discs (vertebrae), heart valves etc. Collagen, particularly collagen nanoparticles, is effectively used in various types of drug administration, because it is biodegradable and does not act as an antigenic agent or cause allergic reactions.

Theophylline is one of the most commonly used medications in bronchial asthma due to its relatively low price, availability, and effectiveness.

Thus, theophylline-loaded collagen nanoparticles to solve the aforementioned problems are desired.

### SUMMARY OF THE INVENTION

The present subject matter relates to theophylline-loaded collagen nanoparticles having an average size ranging from about 30 nm to about 40 nm. The theophylline-loaded collagen nanoparticles enhance the therapeutic effect of theophylline against bronchial asthma.

In another embodiment, the present subject matter relates to a pharmaceutical composition comprising a nanocomposite and a pharmaceutically acceptable carrier, the nanocomposite comprising theophylline-loaded collagen nanoparticles, the nanocomposite having a particle size ranging from about 30 nm to about 40 nm.

In a further embodiment, the present subject matter relates to a nanocomposite comprising collagen nanoparticles loaded with theophylline, the nanocomposite having a particle size ranging from about 30 nm to about 40 nm, wherein collagen in the collagen nanoparticles is derived from scales of mullet fish.

These and other features of the present subject matter will become readily apparent upon further review of the following specification.

### BRIEF DESCRIPTION OF THE DRAWINGS

FIGS. 1A-1D show Fourier transform infrared (FTIR) spectra of (A) collagen, (B) collagen nanoparticles, (C) theophylline and (D) and theophylline loaded with collagen nanoparticles.

FIGS. 2A-2C depict scanning electron microscopy (SEM) images of collagen fibers.

FIGS. 3A-3B depict transmission electron microscope (TEM) images of (A) collagen nanoparticles and (B) theophylline-loaded collagen nanoparticles.

FIG. 4 is a schematic diagram depicting a method used for inducing asthma in rats and treatment of the asthma-induced rats.

FIG. 5 is a graph showing changes of IgE level in serum from studied groups. (Means with the same letter are not significantly different from each other (P<0.05)).

FIG. 6 is a graph showing airway reactivity to cumulative doses of acetylcholine in in vitro tracheal strips from studied groups. (Means with the same letter are not significantly different from each other (P<0.001)).

### DETAILED DESCRIPTION

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

“Subject” or “patient” as used herein refers to any animal classified as a mammal, including humans, domestic and farm animals, and zoo, sports, and pet companion animals such as household pets and other domesticated animals such as, but not limited to, cattle, sheep, ferrets, swine or pigs, horses, camels, poultry, rabbits, goats, dogs, cats, and the like.

As used herein, the term “providing” an agent is used to include “administering” the agent to a subject.

As used herein, a “carrier” includes any and all solvents, dispersion media, vehicles, coatings, diluents, isotonic and absorption delaying agents, buffers, carrier solutions, suspensions, colloids, excipients, and the like.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In an embodiment, the present subject matter relates to a nanocomposite comprising collagen nanoparticles loaded with theophylline. In an embodiment, the nanoparticles are generally spherical and can have an average particle diameter ranging from about 30 nm to about 40 nm. The collagen nanoparticles can be derived from a marine source, such as mullet fish.

As described herein, the nanocomposite provides improved therapeutic efficacy in treating asthma compared to that achieved from use of theophylline alone. In an embodiment, the nanocomposite can provide improved efficacy with lower doses of theophylline. In an embodiment, the nanocomposite is administered intratracheally.

As described herein, encapsulation of theophylline into collagen nanoparticles or the nanocomposite formulation, as described herein, can improve the therapeutic efficacy of theophylline by enhancing its bioavailability.

In an embodiment, the nanocomposite can have an average particle size ranging from about 30 nm to about 40 nm. In other embodiments, the nanocomposite can have an average particle diameter of about 30 nm, about 31 nm, about 32 nm, about 33 nm, about 34 nm, about 35 nm, about 36 nm, about 37 nm, about 38 nm, about 39 nm, and about 40 nm.

In one embodiment, the present subject matter relates to a method of synthesizing a nanocomposite including theophylline-loaded collagen nanoparticles comprising extracting collagen from scales of mullet fish, providing an aqueous collagen solution, adding dimethylsulfoxide (DMSO) to the aqueous collagen solution to provide a first mixture, adding theophylline to the mixture to provide a second mixture, and adding glutaraldehyde to the second mixture to provide the nanocomposite including theophylline-loaded collagen nanoparticles.

It is believed that use of dimethylsulfoxide (DMSO) as the solvent in the preparation of the drug-loaded collagen nanoparticles results in a decreased particle size compared to nanoparticles obtained using prior methods. The decreased particle size provides increased surface area and, thus, enhanced pharmaceutical application of these nanoparticles. In addition, nanoparticle uptake by the cells is accelerated and, subsequently, the nanoparticles are released easily from the body.

An embodiment of the present subject matter is directed to a pharmaceutical composition comprising the nanocomposite and a pharmaceutically acceptable carrier.

An embodiment of the present subject matter is directed to a method of making a pharmaceutical composition including mixing the nanocomposite with a pharmaceutically acceptable carrier. For example, the method of making a pharmaceutical composition can include mixing the nanocomposite under sterile conditions with a pharmaceutically acceptable carrier with preservatives, buffers, and/or propellants to create the pharmaceutical composition.

To prepare the pharmaceutical composition, the nanocomposite, as the active ingredient, is intimately admixed with a pharmaceutically acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The present compositions can be in unit dosage forms such as tablets, pills, capsules, powders, granules, ointments, sterile parenteral solutions or suspensions, metered aerosol or liquid sprays, drops, ampules, auto-injector devices or suppositories, for oral parenteral, intranasal, sublingual or rectal administration, or for administration by injection, inhalation or insufflation. The nanocomposite can be mixed under sterile conditions with a pharmaceutically acceptable carrier and, if required, any needed preservatives, buffers, or propellants. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose. A therapeutically effective amount of the nanocomposite or an amount effective to treat a disease, such as bronchial asthma, may be determined initially from the Examples described herein and adjusted for specific targeted diseases using routine methods.

The nanocomposite can be administered to a subject in need thereof. In an embodiment, the nanocomposite can be administered to a subject in need thereof to treat bronchial asthma.

An embodiment of the present subject matter is directed to a method of treating and preventing bronchial asthma in a subject, comprising administering to a subject in need thereof a therapeutically effective amount of the pharmaceutical composition according to the present subject matter. In an embodiment, a therapeutically effective amount of the pharmaceutical composition can range from about 1 mg/kg/day to about 20 mg/kg/day.

The nanocomposite or pharmaceutical compositions thereof can be administered to a subject by any suitable route. For example, the compositions can be administered orally (including bucally and sublingually), nasally, rectally, intracisternally, intra vaginally, intraperitoneally, topically, transdermally (as by powders, ointments, or drops), and/or parenterally. As used herein, “parenteral” administration refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation may also be contemplated, including, for example, embedding a composition of the disclosure in the body such as, for example, in a tissue, in the abdominal cavity, under the splenic capsule, brain, or in the cornea.

The present teachings are illustrated by the following examples.

### EXAMPLES

### Example 1

### Preparation of Fish Scales and Collagen Extraction

Scales of grey mullet fish were isolated by hand and cleared with distilled water. The samples were dried, placed in polybags, and kept at 25° C. until use. Non-collagenous proteins and pigments were removed from the fish scales with 0.1N NaOH for two days and then cleared with distilled water. The samples were then extracted for two days with an acetic acid concentration (0.50 M) and then homogenized for 3 hours. The supernatants were removed, and the remaining solution including collagen was filtrated.

### Example 2

### Preparation of Collagen Nanoparticles and Drug Loading

The nanoprecipitation technique was used to create collagen nanoparticles using non-solvent Dimethylsulfoxide (DMSO). DMSO was added to an aqueous collagen solution utilizing a burette with free flow under stirring, which resulted in protein denaturation from stretched to coil conformational change. Theophylline was added slowly to the aqueous solution. To induce particle cross-linking, glutaraldehyde was added with stirring. The solution of drug-encapsulated nanoparticles was centrifuged, lyophilized, and stored for later use.

### Example 3

### FTIR Spectrum Analysis

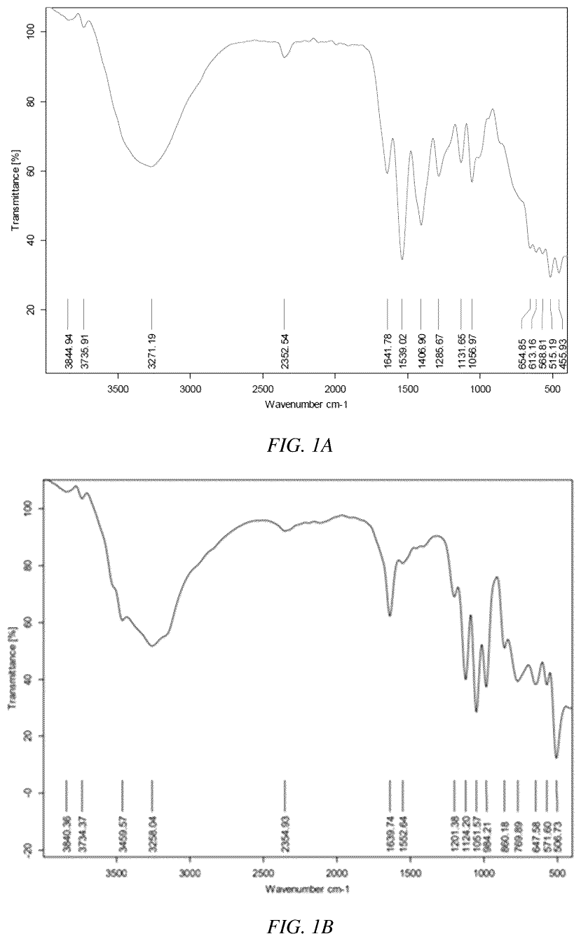

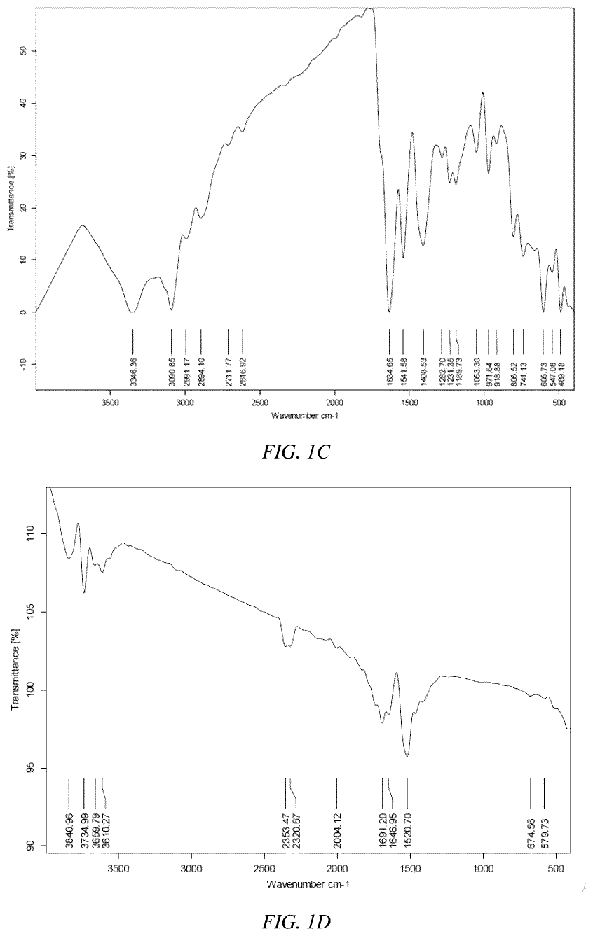

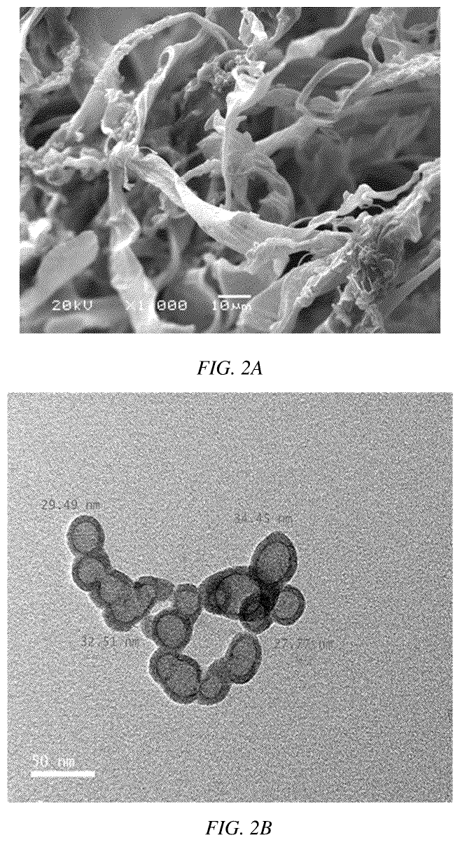

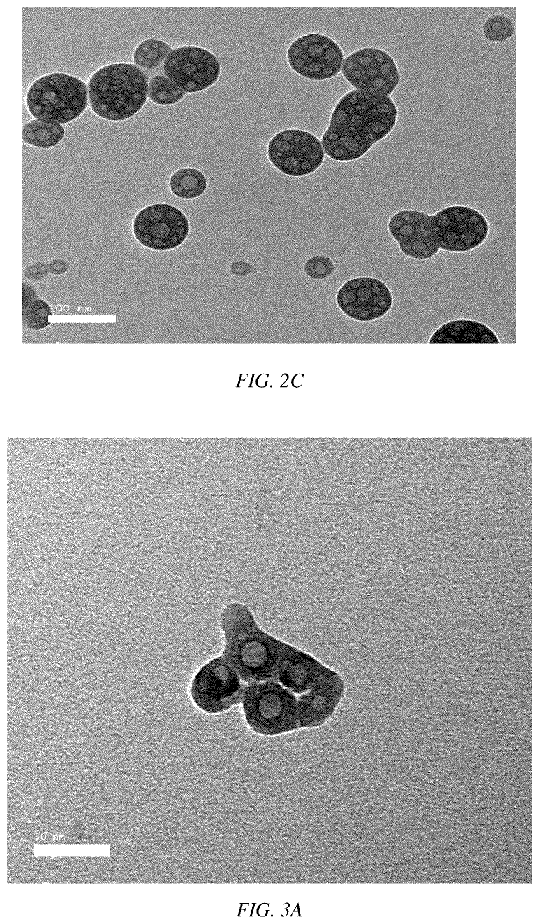

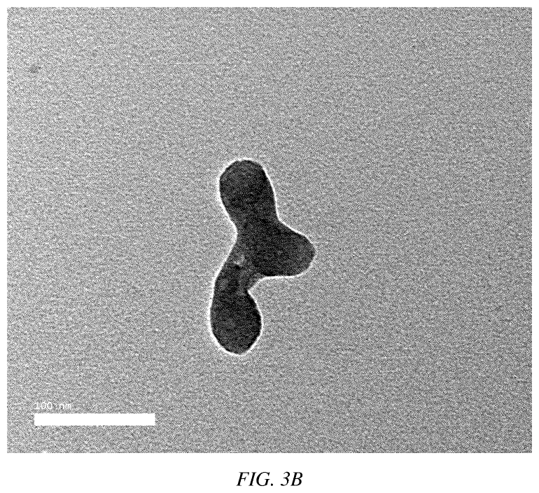

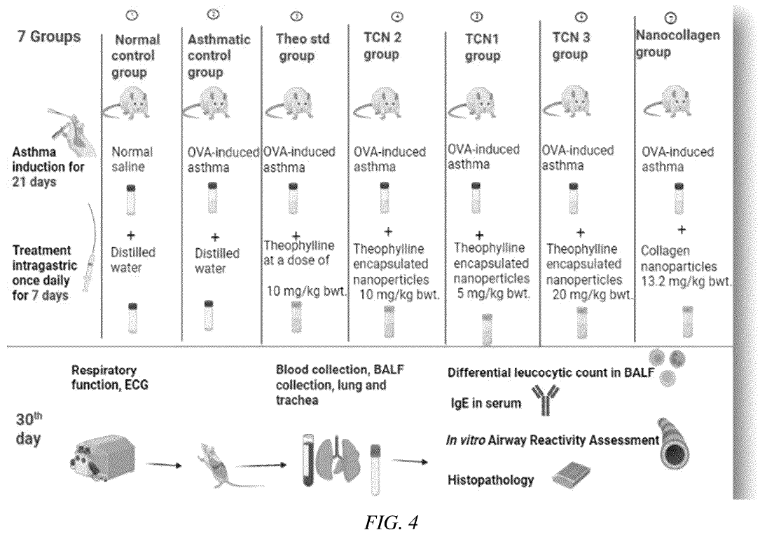

Fourier transform infrared (FTIR) spectroscopy of a freeze-dried sample of collagen, collagen nanoparticles, theophylline, and theophylline loaded with collagen nanoparticles (TCN) was carried out using Shimadzu FTIR 8400S, Japan at SRTA-City, Egypt (FIGS. 1A-1D). Sample (10 mg) was mixed with 100 mg of dried potassium bromide (KBr) and compressed to prepare a disc of 10 mm diameter. Spectra were recorded between 4000 and 500 cm⁻¹.

### Example 4

### Efficacy Assessment of Theophylline-Loaded Collagen Nanoparticles on Experimentally-Induced Bronchial Asthma in Rats

Forty-nine male rats were divided into the following seven groups:
1) Group I-Control;
2) Group II-Asthmatic-Control;
3) Group III-Theophylline-Standard;
4) Group IV-Theophylline-loaded Collagen Nanoparticles-TCN-2;
5) Group V-Theophylline-loaded Collagen Nanoparticles-TCN-1;
6) Group VI-Theophylline-loaded Collagen Nanoparticles-TCN-3; and
7) Group VI-Collagen Nanoparticles.

Asthma was induced in the rats of Groups 2-7 using Ovalbumin (OVA) for twenty-one days. Group 2 was administered theophylline alone at a dose of 10 mg/kg/bwt for seven days. Group 3 was administered theophylline-loaded collagen nanoparticles at a dose of 10 mg/kg/bwt for seven days. Group 4 was administered theophylline-loaded collagen nanoparticles at a dose of 5 mg/kg/bwt for seven days. Group 5 was administered theophylline-loaded collagen nanoparticles at a dose of 20 mg/kg/bwt for seven days. Group 6 was administered collagen nanoparticles alone at a dose of 13.2 mg/kg/bwt for seven days.

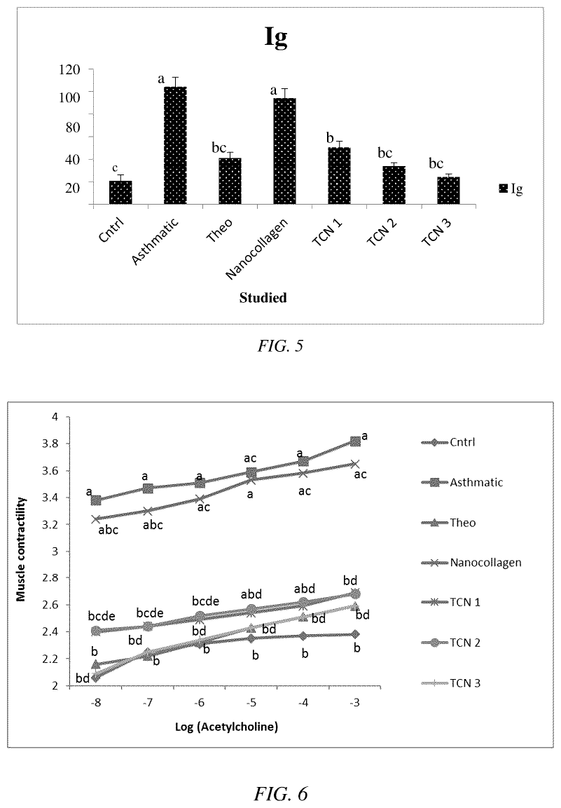

Administration of theophylline-encapsulated nanoparticles in bronchial asthma induced in rats resulted in improved respiratory function tests, a marked decrease in eosinophils and differential leucocytic count, a decline in immunoglobulin E (FIG. 5) and a decrease in invitro reactivity assessment (FIG. 6) in tracheal muscle compared to the theophylline group. Histopathological examination showed remarkable improvement in theophylline-loaded collagen nanoparticles-treated groups in comparison to the theophylline-treated group.

It is to be understood that the nanocomposite is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. (canceled)

2. (canceled)

3. (canceled)

4. (canceled)

5. (canceled)

6. A method of synthesizing a nanocomposite, comprising:
extracting collagen from scales of mullet fish;
providing an aqueous collagen solution;
adding dimethyl sulfoxide (DMSO) to the aqueous collagen solution to provide a first mixture;
adding theophylline to the first mixture to provide a second mixture; and
adding glutaraldehyde to the second mixture to provide the nanocomposite including theophylline-loaded collagen nanoparticles.

7. A nanocomposite prepared according to the method of claim 6, the nanocomposite having a particle size ranging from about 30 nm to about 40 nm.

8. A pharmaceutical composition comprising the nanocomposite of claim 7 and a pharmaceutically acceptable carrier.

9. A method of treating bronchial asthma in a subject, comprising administering to a subject in need thereof a therapeutically effective amount of the pharmaceutical composition of claim 8.

10. (canceled)

11. (canceled)

12. (canceled)

13. (canceled)
